(19) United States
(12) Patent Application Publication
(10) Pub. No.: US 2022/0062332 A1
(43) Pub. Date: Mar. 3, 2022

(54) Method of Treatment for HIV Infection

(71) Applicant: Shawne Forrest, Dallas, TX (US)

(72) Inventor: Shawne Forrest, Dallas, TX (US)

(21) Appl. No.: 17/008,126

(22) Filed: Aug. 31, 2020

(51) Int. Cl. A61K 33/28; A61K 47/12; A61K 33/10; A61K 39/21; A61K 39/108; A61K 35/14; A61K 47/44; A61K 31/05

(52) U.S. Cl. CPC A61K 33/28; A61K 47/12; A61K 33/10; A61K 31/05; A61K 39/0258; A61K 35/14; A61K 47/44; A61K 39/21

(57) ABSTRACT

A method of treating an HIV infected human is carried out by injecting into the lymph nodes of the HIV infected human a composition comprising an amount of calcium bicarbonate dissolved or dispersed in a carrier fluid capable of being absorbed into the CD4+ cells (T cells) of the lymph nodes. This forms modified CD4+ cells that present antigens that can be then targeted and destroyed by the HIV infected human's natural immune system so that the CD4+ cells of the HIV infected are destroyed, thus preventing replication of the HIV virus. This may be used in conjunction with reducing the HIV in the blood by a liver treatment using a composition comprising HIV virus, blood lysine, and E. coli and/or dialysis using centrifuging and microfiltration.

### TECHNICAL FIELD

The invention relates to methods of treatment of HIV infected humans and to prevent the transmission of HIV.

### BACKGROUND

The human immunodeficiency viruses (HIV) that causes acquired immunodeficiency syndrome (AIDS) is a virus that is typically transmitted through the exchange of bodily fluids from the infected individual. It is typically transmitted through unprotected sexual contact, although it may be transmitted through blood transfusions or the shared use of hypodermic needles. HIV infects cells of the immune system, such CD4+ cells (T cells), that help to fight off infections. The HIV infection reduces the number of such cells so that the infected person's immune system is weakened, ultimately leading to the AIDS condition where the body can no longer effectively fight off infections and disease, eventually resulting in the death of the infected individual.

Accordingly, a need exists for the treatment of those individuals infected with HIV and for methods of preventing transmission of HIV from an infected individual to a non-infected individual.

### DETAILED DESCRIPTION

One of the earliest treatments for gonorrhea involved injecting mercury directly into the urethra of the infected person. By changing this chemical formula to an organomercury compound, such as thimerosal, in combination with other compounds, the transmission of HIV can be reduced or eliminated.

The prophylactic treatment composition to prevent the transmission of HIV includes an amount of an organomercury compound, such as thimerosal. The organomercury compound or thimerosal may be present in the composition in an amount of at least, equal to, and/or between any two of 0.001 wt. %, 0.002 wt. %, 0.003 wt. %, 0.004 wt. %, 0.005 wt. %, 0.006 wt. %, 0.007 wt. %, 0.008 wt. %, 0.009 wt. %, 0.01 wt. %, 0.02 wt. %, 0.03 wt. %, 0.04 wt. %, 0.05 wt. %, 0.06 wt. %, 0.07 wt. %, 0.08 wt. %, 0.09 wt. %, 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1.0 wt. %, 1.1 wt. %, 1.2 wt. %, 1.3 wt. %, 1.4 wt. %, 1.5 wt. %, 1.6 wt. %, 1.7 wt. %, 1.8 wt. %, 1.9 wt. %, 2.0 wt. %, 2.1 wt. %, 2.2 wt. %, 2.3 wt. %, 2.4 wt. %, 2.5 wt. %, 2.6 wt. %, 2.7 wt. %, 2.8 wt. %, 2.9 wt. %, 3.0 wt. %, 3.1 wt. %, 3.2 wt. %, 3.3 wt. %, 3.4 wt. %, 3.5 wt. %, 3.6 wt. %, 3.7 wt. %, 3.8 wt. %, 3.9 wt. %, 4.0 wt. %, 4.1 wt. %, 4.2 wt. %, 4.3 wt. %, 4.4 wt. %, 4.5 wt. %, 4.6 wt. %, 4.7 wt. %, 4.8 wt. %, 4.9 wt. %, 5.0 wt. %, 5.5 wt. %, 6.0 wt. %, 6.5 wt. %, 7.5 wt. %, 8.0 wt. %, 8.5 wt. %, 9.0 wt. %, 9.5 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, 30 wt. %, 31 wt. %, 32 wt. %, 33 wt. %, 34 wt. %, 35 wt. %, 36 wt. %, 37 wt. %, 38 wt. %, 39 wt. %, 40 wt. %, 41 wt. %, 42 wt. %, 43 wt. %, 44 wt. %, 45 wt. %, 46 wt. %, 47 wt. %, 48 wt. %, 49 wt. %, 50 wt. %, 51 wt. %, 52 wt. %, 53 wt. %, 54 wt. %, 55 wt. %, 56 wt. %, 57 wt. %, 58 wt. %, 59 wt. %, 60 wt. % by total weight of the prophylactic treatment composition.

It should be noted in the description, if a numerical value, concentration or range is presented, each numerical value should be read once as modified by the term “about” (unless already expressly so modified), and then read again as not so modified unless otherwise indicated in context. Also, in the description, it should be understood that an amount range listed or described as being useful, suitable, or the like, is intended that any and every value within the range, including the end points, is to be considered as having been stated. For example, “a range of from 1 to 10” is to be read as indicating each and every possible number along the continuum between about 1 and about 10. Thus, even if specific points within the range, or even no point within the range, are explicitly identified or referred to, it is to be understood that the inventor appreciates and understands that any and all points within the range are to be considered to have been specified, and that inventor possesses the entire range and all points within the range, including smaller ranges within the larger ranges.

Further, the prophylactic treatment composition includes an ascorbic acid and/or an ascorbic acid salt component. The ascorbic acid and/or ascorbic acid salt may be present in the treatment composition in an amount of from at least, equal to, and/or between any two of 0.001 wt. %, 0.002 wt. %, 0.003 wt. %, 0.004 wt. %, 0.005 wt. %, 0.006 wt. %, 0.007 wt. %, 0.008 wt. %, 0.009 wt. %, 0.01 wt. %, 0.02 wt. %, 0.03 wt. %, 0.04 wt. %, 0.05 wt. %, 0.06 wt. %, 0.07 wt. %, 0.08 wt. %, 0.09 wt. %, 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1.0 wt. %, 1.1 wt. %, 1.2 wt. %, 1.3 wt. %, 1.4 wt. %, 1.5 wt. %, 1.6 wt. %, 1.7 wt. %, 1.8 wt. %, 1.9 wt. %, 2.0 wt. %, 2.1 wt. %, 2.2 wt. %, 2.3 wt. %, 2.4 wt. %, 2.5 wt. %, 2.6 wt. %, 2.7 wt. %, 2.8 wt. %, 2.9 wt. %, 3.0 wt. %, 3.1 wt. %, 3.2 wt. %, 3.3 wt. %, 3.4 wt. %, 3.5 wt. %, 3.6 wt. %, 3.7 wt. %, 3.8 wt. %, 3.9 wt. %, 4.0 wt. %, 4.1 wt. %, 4.2 wt. %, 4.3 wt. %, 4.4 wt. %, 4.5 wt. %, 4.6 wt. %, 4.7 wt. %, 4.8 wt. %, 4.9 wt. %, 5.0 wt. %, 5.5 wt. %, 6.0 wt. %, 6.5 wt. %, 7.5 wt. %, 8.0 wt. %, 8.5 wt. %, 9.0 wt. %, 9.5 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, 30 wt. %, 31 wt. %, 32 wt. %, 33 wt. %, 34 wt. %, 35 wt. %, 36 wt. %, 37 wt. %, 38 wt. %, 39 wt. %, 40 wt. %, 41 wt. %, 42 wt. %, 43 wt. %, 44 wt. %, 45 wt. %, 46 wt. %, 47 wt. %, 48 wt. %, 49 wt. %, 50 wt. %, 51 wt. %, 52 wt. %, 53 wt. %, 54 wt. %, 55 wt. %, 56 wt. %, 57 wt. %, 58 wt. %, 59 wt. %, 60 wt. % by total weight of the prophylactic treatment composition.

The prophylactic treatment composition further includes citric acid and/or a citric acid salt. The citric acid and/or a citric acid salt may be present in the treatment composition in an amount of from at least, equal to, and/or between any two of 0.001 wt. %, 0.002 wt. %, 0.003 wt. %, 0.004 wt. %, 0.005 wt. %, 0.006 wt. %, 0.007 wt. %, 0.008 wt. %, 0.009 wt. %, 0.01 wt. %, 0.02 wt. %, 0.03 wt. %, 0.04 wt. %, 0.05 wt. %, 0.06 wt. %, 0.07 wt. %, 0.08 wt. %, 0.09 wt. %, 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1.0 wt. %, 1.1 wt. %, 1.2 wt. %, 1.3 wt. %, 1.4 wt. %, 1.5 wt. %, 1.6 wt. %, 1.7 wt. %, 1.8 wt. %, 1.9 wt. %, 2.0 wt. %, 2.1 wt. %, 2.2 wt. %, 2.3 wt. %, 2.4 wt. %, 2.5 wt. %, 2.6 wt. %, 2.7 wt. %, 2.8 wt. %, 2.9 wt. %, 3.0 wt. %, 3.1 wt. %, 3.2 wt. %, 3.3 wt. %, 3.4 wt. %, 3.5 wt. %, 3.6 wt. %, 3.7 wt. %, 3.8 wt. %, 3.9 wt. %, 4.0 wt. %, 4.1 wt. %, 4.2 wt. %, 4.3 wt. %, 4.4 wt. %, 4.5 wt. %, 4.6 wt. %, 4.7 wt. %, 4.8 wt. %, 4.9 wt. %, 5.0 wt. %, 5.5 wt. %, 6.0 wt. %, 6.5 wt. %, 7.5 wt. %, 8.0 wt. %, 8.5 wt. %, 9.0 wt. %, 9.5 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, 30 wt. %, 31 wt. %, 32 wt. %, 33 wt. %, 34 wt. %, 35 wt. %, 36 wt. %, 37 wt. %, 38 wt. %, 39 wt. %, 40 wt. %, 41 wt. %, 42 wt. %, 43 wt. %, 44 wt. %, 45 wt. %, 46 wt. %, 47 wt. %, 48 wt. %, 49 wt. %, 50 wt. %, 51 wt. %, 52 wt. %, 53 wt. %, 54 wt. %, 55 wt. %, 56 wt. %, 57 wt. %, 58 wt. %, 59 wt. %, 60 wt. % by total weight of the prophylactic treatment composition.

These compounds can be combined in different proportions in an aqueous solution or other suitable carrier liquid or compound to form the treatment composition. The water or carrier liquid or compound may make up from 40 wt. % to 99.997 wt. % of the prophylactic treatment composition.

In certain instances, the prophylactic treatment composition has a pH that effectively lowers the pH of bodily fluids in which the composition is present to a pH of from 4.5 or 4.0 or less. HIV survives in an optimal level pH of from 7 to 8. Lowering the pH of the bodily fluids to 4.5 or 4.0 or less makes survival of the virus in such bodily fluids unlikely.

A dose of the treatment composition is then injecting into at least one of an anatomical feature of a urethra, a seminal tract, a prostate gland, a vagina, and a rectum of an HIV-infected human. For males, injection of the composition into the urethra, seminal tract, and/or prostate gland would result in the HIV-positive seminal fluids to become laced with the treatment composition, which may be reduced to a pH of 4.5, 4.0 or less.

Moreover, introducing such composition in the vagina or rectum of an infected or non-infected person, may also prevent or reduce the transmission of the HIV virus. The presence of the prophylactic treatment composition in bodily fluids in such areas will kill the HIV virus that is either initially present or is subsequently introduced.

In women, in conjunction with a healthy vagina or rectum that has large amounts of Lactobacillus acidophilus, this should also facilitate reducing the transmission of HIV due to a lower pH of 4.5 in conjunction with this compound, since the pH will be lowered by the compound and the HIV virus dies at pH of around 4. Many other sexually transmitted viruses, such as the herpes simplex viruses also cannot survive at such low pH levels. The treatment composition could also be administered once or twice as it relates to HPV in woman. The compound can be administered to the Grafenberg area or “G spot” of a woman's vagina, as the HPV virus usually remains localized in these areas in HPV positive women.

In another aspect of the invention, a treatment for HIV infected individuals involves modifying the person's immune system by keeping it in a hyper immune state and/or killing off those immune system cells that allow the HIV virus to replicate. There is evidence that exposure to bee venom over time can result in an immune response that protects against future venom exposure. This has been observed in certain indigenous tribes in Africa when harvesting honey wherein those harvesting honey from beehives do not suffer any harm after being stung repeatedly by honeybees. The present invention for the treatment of HIV has similarities to this.

Because the HIV virus is good at hiding in the immune system, the first step is to control the spread of the HIV virus in the infected person's system. This can be achieved by exposing the infected person's liver to a liver treatment composition that is ingested or introduced into the digestive system. The liver treatment composition is comprised of an amount of HIV virus, blood lysine, and Escherichia coli (E. coli) in a carrier fluid or compound, which may be an aqueous fluid or non-aqueous fluid or compound.

The HIV virus may be present in the liver treatment composition in an amount of from at least, equal to, and/or between any two of 0.0001 wt. %, 0.0002 wt. %, 0.0003 wt. %, 0.0004 wt. %, 0.0005 wt. %, 0.0006 wt. %, 0.0007 wt. %, 0.0008 wt. %, 0.0009 wt. %, 0.001 wt. %, 0.002 wt. %, 0.003 wt. %, 0.004 wt. %, 0.005 wt. %, 0.006 wt. %, 0.007 wt. %, 0.008 wt. %, 0.009 wt. %, 0.01 wt. %, 0.02 wt. %, 0.03 wt. %, 0.04 wt. %, 0.05 wt. %, 0.06 wt. %, 0.07 wt. %, 0.08 wt. %, 0.09 wt. %, 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1.0 wt. %, 1.1 wt. %, 1.2 wt. %, 1.3 wt. %, 1.4 wt. %, 1.5 wt. %, 1.6 wt. %, 1.7 wt. %, 1.8 wt. %, 1.9 wt. %, 2.0 wt. %, 2.1 wt. %, 2.2 wt. %, 2.3 wt. %, 2.4 wt. %, 2.5 wt. %, 2.6 wt. %, 2.7 wt. %, 2.8 wt. %, 2.9 wt. %, 3.0 wt. %, 3.1 wt. %, 3.2 wt. %, 3.3 wt. %, 3.4 wt. %, 3.5 wt. %, 3.6 wt. %, 3.7 wt. %, 3.8 wt. %, 3.9 wt. %, 4.0 wt. %, 4.1 wt. %, 4.2 wt. %, 4.3 wt. %, 4.4 wt. %, 4.5 wt. %, 4.6 wt. %, 4.7 wt. %, 4.8 wt. %, 4.9 wt. %, 5.0 wt. %, 5.5 wt. %, 6.0 wt. %, 6.5 wt. %, 7.5 wt. %, 8.0 wt. %, 8.5 wt. %, 9.0 wt. %, 9.5 wt. %, 10 wt. %, 11 wt. %, 12 wt. %, 13 wt. %, 14 wt. %, 15 wt. %, 16 wt. %, 17 wt. %, 18 wt. %, 19 wt. %, and 20 wt. % by weight of the liver treatment composition.

The blood lysine may be present in the liver treatment composition in an amount of from at least, equal to, and/or between any two of 0.001 wt. %, 0.002 wt. %, 0.003 wt. %, 0.004 wt. %, 0.005 wt. %, 0.006 wt. %, 0.007 wt. %, 0.008 wt. %, 0.009 wt. %, 0.01 wt. %, 0.02 wt. %, 0.03 wt. %, 0.04 wt. %, 0.05 wt. %, 0.06 wt. %, 0.07 wt. %, 0.08 wt. %, 0.09 wt. %, 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1.0 wt. %, 1.1 wt. %, 1.2 wt. %, 1.3 wt. %, 1.4 wt. %, 1.5 wt. %, 1.6 wt. %, 1.7 wt. %, 1.8 wt. %, 1.9 wt. %, 2.0 wt. %, 2.1 wt. %, 2.2 wt. %, 2.3 wt. %, 2.4 wt. %, 2.5 wt. %, 2.6 wt. %, 2.7 wt. %, 2.8 wt. %, 2.9 wt. %, 3.0 wt. %, 3.1 wt. %, 3.2 wt. %, 3.3 wt. %, 3.4 wt. %, 3.5 wt. %, 3.6 wt. %, 3.7 wt. %, 3.8 wt. %, 3.9 wt. %, 4.0 wt. %, 4.1 wt. %, 4.2 wt. %, 4.3 wt. %, 4.4 wt. %, 4.5 wt. %, 4.6 wt. %, 4.7 wt. %, 4.8 wt. %, 4.9 wt. %, 5.0 wt. %, 5.5 wt. %, 6.0 wt. %, 6.5 wt. %, 7.5 wt. %, 8.0 wt. %, 8.5 wt. %, 9.0 wt. %, 9.5 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, 30 wt. %, 31 wt. %, 32 wt. %, 33 wt. %, 34 wt. %, 35 wt. %, 36 wt. %, 37 wt. %, 38 wt. %, 39 wt. %, 40 wt. %, 41 wt. %, 42 wt. %, 43 wt. %, 44 wt. %, 45 wt. %, 46 wt. %, 47 wt. %, 48 wt. %, 49 wt. %, 50 wt. %, 51 wt. %, 52 wt. %, 53 wt. %, 54 wt. %, 55 wt. %, 56 wt. %, 57 wt. %, 58 wt. %, 59 wt. %, 60 wt. % by total weight of the liver treatment composition.

The E. coli may be present in the liver treatment composition in an amount of from at least, equal to, and/or between any two of 0.0001 wt. %, 0.0002 wt. %, 0.0003 wt. %, 0.0004 wt. %, 0.0005 wt. %, 0.0006 wt. %, 0.0007 wt. %, 0.0008 wt. %, 0.0009 wt. %, 0.001 wt. %, 0.002 wt. %, 0.003 wt. %, 0.004 wt. %, 0.005 wt. %, 0.006 wt. %, 0.007 wt. %, 0.008 wt. %, 0.009 wt. %, 0.01 wt. %, 0.02 wt. %, 0.03 wt. %, 0.04 wt. %, 0.05 wt. %, 0.06 wt. %, 0.07 wt. %, 0.08 wt. %, 0.09 wt. %, 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1.0 wt. %, 1.1 wt. %, 1.2 wt. %, 1.3 wt. %, 1.4 wt. %, 1.5 wt. %, 1.6 wt. %, 1.7 wt. %, 1.8 wt. %, 1.9 wt. %, 2.0 wt. %, 2.1 wt. %, 2.2 wt. %, 2.3 wt. %, 2.4 wt. %, 2.5 wt. %, 2.6 wt. %, 2.7 wt. %, 2.8 wt. %, 2.9 wt. %, 3.0 wt. %, 3.1 wt. %, 3.2 wt. %, 3.3 wt. %, 3.4 wt. %, 3.5 wt. %, 3.6 wt. %, 3.7 wt. %, 3.8 wt. %, 3.9 wt. %, 4.0 wt. %, 4.1 wt. %, 4.2 wt. %, 4.3 wt. %, 4.4 wt. %, 4.5 wt. %, 4.6 wt. %, 4.7 wt. %, 4.8 wt. %, 4.9 wt. %, 5.0 wt. %, 5.5 wt. %, 6.0 wt. %, 6.5 wt. %, 7.5 wt. %, 8.0 wt. %, 8.5 wt. %, 9.0 wt. %, 9.5 wt. %, 10 wt. %, 11 wt. %, 12 wt. %, 13 wt. %, 14 wt. %, 15 wt. %, 16 wt. %, 17 wt. %, 18 wt. %, 19 wt. %, and 20 wt. % by weight of the liver treatment composition.

The carrier fluid or compound, which may be an aqueous fluid or non-aqueous fluid or compound may make up from 40 wt. % to 99.997 wt. % of the treatment composition. In some embodiments of the liver treatment composition, all or a portion of the carrier fluid may include honey. Honey may provide a higher immune response in a similar manner to the way latent botulism and tetanus immunity develops.

The liver is exposed to the treatment composition via the digestive system so that increased production of antibodies in the infected human is facilitated. In doing this, these antibodies will facilitate neutralizing the HIV virus. Once the HIV levels have become undetectable you will allow for substantial amounts of antibodies to be created and remain in the system as compared to the virus in the blood stream and lymph nodes. This will allow for any free viruses that have not been infective to neutralized by the heightened immunity caused by exposure of the liver to the treatment composition containing the HIV virus, blood lysine, and E. coli.

In certain embodiments, this technique can be used alone or in conjunction with dialysis, followed by centrifugation in combination with microfiltration so that the HIV present in the blood is reduced or eliminated through such techniques so that the HIV is undetectable in the blood, i.e., less than 200, 100 or 50 HIV copies/ml of blood. Likewise, the dialysis with centrifugation and microfiltration without the liver treatment may also be used to reduce the HIV virus in the blood to undetectable levels. The waste fluid or filtrate containing the HIV from the dialysis is collected and further centrifuged in centrifuge tubes that utilize a microfilter using a filter media capable of filtering particles of from 2.5 to 3 microns or greater. In certain instances, such microfilitration may also include magnetization to help retain the infected CD4+ cells during filtration because such HIV infected cells have exhibited a change in polarity. The dialysis treatments may be carried out for a period of from 4 to 6 hours or more.

In certain aspects, dialysis using centrifugation and microfiltration, as described above, will result in both the virus and antibodies being concentrated and collected in the filtrate that pass through the microfilter. The filtrate can be kept for a period of time (e.g., 1-2 days) to allow the HIV material to become inactive. The inactive HIV/antibody material can then be used itself in a composition for inoculating others or be reintroduced into the person that is being treated to further boost their immunity.

The process of reducing the HIV in the blood, using the liver treatment and/or dialysis, to undetectable levels may take several days to several weeks or even months.

With or without exposing of the liver to the liver treatment composition and/or dialysis, a further treatment is used to target the lymph nodes. Cells infected with the HIV virus in the lymph nodes do not present antigens. Therefore, in order to reduce or eliminate the infection in the lymph nodes, it is necessary to modify the CD4+ cells (T cells) of the lymph nodes so that they present antigens so they can be destroyed. This can be done by targeting the lymph nodes with a direct injection of a further lymph node treatment composition. This lymph node treatment composition contains an amount of calcium bicarbonate dissolved in an aqueous carrier fluid that is capable of being absorbed into the CD4+ cells (T cells). Such modified CD4+ cells present antigens that can be then targeted and destroyed by the HIV infected human's natural immune system so that the CD4+ cells of the HIV infected human are destroyed, thus preventing replication of the HIV virus.

The calcium bicarbonate may be present in the lymph node treatment composition in an amount of from at least, equal to, and/or between any two of 0.001 wt. %, 0.002 wt. %, 0.003 wt. %, 0.004 wt. %, 0.005 wt. %, 0.006 wt. %, 0.007 wt. %, 0.008 wt. %, 0.009 wt. %, 0.01 wt. %, 0.02 wt. %, 0.03 wt. %, 0.04 wt. %, 0.05 wt. %, 0.06 wt. %, 0.07 wt. %, 0.08 wt. %, 0.09 wt. %, 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1.0 wt. %, 1.1 wt. %, 1.2 wt. %, 1.3 wt. %, 1.4 wt. %, 1.5 wt. %, 1.6 wt. %, 1.7 wt. %, 1.8 wt. %, 1.9 wt. %, 2.0 wt. %, 2.1 wt. %, 2.2 wt. %, 2.3 wt. %, 2.4 wt. %, 2.5 wt. %, 2.6 wt. %, 2.7 wt. %, 2.8 wt. %, 2.9 wt. %, 3.0 wt. %, 3.1 wt. %, 3.2 wt. %, 3.3 wt. %, 3.4 wt. %, 3.5 wt. %, 3.6 wt. %, 3.7 wt. %, 3.8 wt. %, 3.9 wt. %, 4.0 wt. %, 4.1 wt. %, 4.2 wt. %, 4.3 wt. %, 4.4 wt. %, 4.5 wt. %, 4.6 wt. %, 4.7 wt. %, 4.8 wt. %, 4.9 wt. %, 5.0 wt. %, 5.5 wt. %, 6.0 wt. %, 6.5 wt. %, 7.5 wt. %, 8.0 wt. %, 8.5 wt. %, 9.0 wt. %, 9.5 wt. %, 10 wt. %, 11 wt. %, 12 wt. %, 13 wt. %, 14 wt. %, 15 wt. %, and 16 wt. %, by weight of the lymph node treatment composition.

The balance of the lymph node treatment composition may be made up of the carrier fluid. The carrier fluid may be an aqueous liquid, such as water or saline, with the calcium bicarbonate being dissolved in the aqueous liquid. The carrier fluid may also be a non-aqueous carrier fluid or compound, such as a fat or oil, with calcium bicarbonate being dispersed in the non-aqueous fluid or compound, such as an emulsion with aqueous droplets containing the dissolved calcium bicarbonate being dispersed in the non-aqueous fluid or compound. In certain embodiments, the carrier fluid or compound is a non-aqueous fluid in the form of a fat or oil that may be at least one of an animal derived fat or oil, a plant derived fat or oil, a beef fat or oil, a vegetable fat or oil, a grape leaf oil, and combinations of these. The fat or oil may be an ozonated oil or non-ozonated oil. In certain embodiments, ozonated fats and oils may be used more effectively.

The lymph node treatment composition is injected into the lymph node of the HIV infected person. The injected calcium bicarbonate is targeted by the CD4+ cells. In doing this any survivor infected cells can be targeted to present as acute lymphoma and thus be destroyed by the natural killer T cells and macrophages. It is theorized that the bicarbonate interacts with chloride (Cl⁻) present in the blood, cleaving the DNA of the infected cells so that the mitochondria of the cells cease working. This may allow free antibodies to attract macrophages with additional antigens that will separate the infected cells and non-infected cells.

In certain embodiments, the above-described treatment of injecting the lymph node treatment composition in to the lymph nodes of an HIV infected human may be carried out by first pretreating the lymph node by injecting a fat or oil without any calcium bicarbonate into the lymph node(s) of the HIV infected person. This is to mirror citric acid crystals absorption of free HIV virus in the lymph and attract macrophages. This lymph pretreatment will allow the buildup of antibodies and slow the lymph drainage.

The lymph pretreatment composition may include a fat or oil that may be at least one of an animal derived fat or oil, a plant derived fat or oil, a beef fat or oil, a vegetable fat or oil, a grape leaf oil, and combinations of these. In certain applications, all or a portion of the fat or oil is an ozonated fat or oil. These fats or oils may constitute a source of non-toxic cholesterol.

The lymph pretreatment is then followed by injection of the lymph node treatment composition into the pretreated lymph node. In such cases, the lymph node treatment composition may be a treatment composition using a carrier fluid (e.g., water, saline, etc.) or compound that is free or substantially free (i.e., less than 0.1 wt. %, 0.5 wt. %, or 1 wt. % by weight of treatment composition) of any fats or oils. The lymph node treatment composition without any fats or oils facilitates dissolving of the fats and/or oils of the pretreatment composition. Such lymph node treatment following the lymph pretreatment using the calcium-bicarbonate-free fat or oil may be carried out at least one hour or more after the lymph pretreatment.

As discussed earlier, using the lymph node treatment, any survivor infected cells residing in the lymph system can be targeted to present as acute lymphoma and thus be destroyed by the natural killer T cells and macrophages. It is theorized that the bicarbonate interacts with chloride (Cl⁻) ions present in the blood, cleaving the DNA of the infected cells so that the mitochondria of the cells cease working. This may allow free antibodies to attract macrophages with additional antigens that will separate the infected cells and non-infected cells.

The lymph node treatment, with or without the fat and/or oil lymph pretreatment, may be followed by the injection of non-toxic cannabidiol oil into the treated lymph nodes, which may serve to reverse the effects.

The above described treatments may be carried out repeatedly or periodically over many days, weeks, or months. Because the treatment is aggressive, it may be necessary to isolate the HIV infected person during the treatment as their immune system may be substantially weakened and compromised such that it may be difficult to fight off other infections.

After the final treatment is administered, a plasma of isolated CD4+ cells can be administered to allow for the immune system to rebuild.

While the invention has been shown in some of its forms, it should be apparent to those skilled in the art that it is not so limited, but is susceptible to various changes and modifications without departing from the scope of the invention. Accordingly, it is appropriate that the appended claims be construed broadly and in a manner consistent with the scope of the invention.

I claim:

1. A method for reducing HIV in an HIV infected human to prevent transmission of the HIV or for the treatment of the HIV infected human, the treatment method comprising:
performing at least one of the following:
1) injecting into at least one of an anatomical feature of a urethra, a seminal tract, a prostate gland, a vagina, and a rectum of an HIV-infected human an amount of a first composition comprising an organomercury compound, ascorbic acid and/or an ascorbic acid salt, and citric acid and/or a citric acid salt so that bodily fluids within said anatomical feature contain said composition to reduce the amount of any HIV virus present in the bodily fluids to thereby reduce HIV transmission; and
2) injecting into the lymph nodes of an HIV infected human a second composition comprising an amount of calcium bicarbonate dissolved or dispersed in a carrier fluid capable of being absorbed into the CD4+ cells (T cells) of the lymph nodes to form modified CD4+ cells that present antigens that can be then targeted and destroyed by the HIV infected human's natural immune system so that the CD4+ cells of the HIV infected human are destroyed, thus preventing replication of the HIV virus.

2. The method of claim 1, wherein:
the organomercury compound of the first composition is thimerosal.

3. The method of claim 1, wherein:
the first composition facilitates lowering the pH of the bodily fluids to a pH of from 4.5 or less.

4. The method of claim 1, wherein:
the treatment of injecting into the lymph nodes of an HIV infected human with the second composition further comprises exposing the liver via the digestive system of the HIV infected human to a third composition comprising an amount of HIV virus, blood lysine, and Escherichia coli (E. coli) to facilitate the increased production of antibodies in the infected human as compared to the HIV virus in the blood stream and lymph nodes of the infected human.

5. The method of claim 1, wherein:
the carrier fluid is at least one of a fat and/or oil, an ozonated fat and/or oil, an ozonated animal-derived fat and/or oil, an ozonated beef fat and/or oil, an ozonated plant-derived fat and/or oil, an ozonated grape leaf oil, a non-ozonated fat and/or oil, a non-ozonated animal-derived fat and/or oil, a non-ozonated beef fat and/or oil, a non-ozonated plant-derived fat and/or oil, and a non-ozonated grape leaf oil.

6. The method of claim 1, wherein:
the treatment of injecting into the lymph nodes of an HIV infected human a second composition comprising calcium bicarbonate comprises injecting a fat and/or oil without the calcium bicarbonate into the lymph node followed by the injection of the calcium bicarbonate dissolved in the carrier fluid.

7. The method of claim 6, wherein:
the carrier fluid is free of fats or oils.

8. The method of claim 6, wherein:
the injection of the calcium bicarbonate dissolved in the carrier fluid is carried out at least one hour after injecting the fat and/or oil without the calcium bicarbonate.

9. The method of claim 6, wherein:
the fat and/or oil without the calcium bicarbonate is at least one of an ozonated fat and/or oil, an ozonated animal-derived fat and/or oil, an ozonated beef fat and/or oil, an ozonated plant-derived fat and/or oil, an ozonated grape leaf oil, and non-ozonated grape leaf oil.

10. The method of claim 1, wherein:
a dose of cannabidiol oil is injected into the lymph nodes after injecting the second composition.

11. A method of treating an HIV infected human, the treatment method comprising:
injecting into the lymph nodes of the HIV infected human a composition comprising an amount of calcium bicarbonate dissolved or dispersed in a carrier fluid capable of being absorbed into the CD4+ cells (T cells) of the lymph nodes to form modified CD4+ cells that present antigens that can be then targeted and destroyed by the HIV infected human's natural immune system so that the CD4+ cells of the HIV infected are destroyed, thus preventing replication of the HIV virus.

12. The method of claim 11, further comprising:
exposing the liver via the digestive system of the HIV infected human to a second composition comprising an amount of HIV virus, blood lysine, and Escherichia coli (E. coli) to facilitate the increased production of antibodies in the infected human as compared to the HIV virus in the blood stream and lymph nodes of the infected human.

13. The method of claim 11, wherein:
the carrier fluid is at least one of an aqueous fluid a fat and/or oil, an ozonated fat and/or oil, an ozonated animal-derived fat and/or oil, an ozonated beef fat and/or oil, an ozonated plant-derived fat and/or oil, an ozonated grape leaf oil, a non-ozonated fat and/or oil, a non-ozonated animal-derived fat and/or oil, a non-ozonated beef fat and/or oil, a non-ozonated plant-derived fat and/or oil, and a non-ozonated grape leaf oil.

14. The method of claim 11, wherein:
the treatment of injecting into the lymph nodes of an HIV infected human a composition comprising calcium bicarbonate comprises injecting a fat or oil without the calcium bicarbonate into the lymph node followed by the injection of the calcium bicarbonate dissolved in the carrier fluid.

15. The method of claim 14, wherein:
the carrier fluid is free of fats or oils.

16. The method of claim 14, wherein:
the injection of the calcium bicarbonate dissolved in the carrier fluid is carried out at least one hour after injecting the fat or oil without the calcium bicarbonate.

17. The method of claim 14, wherein:
the fat or oil without the calcium bicarbonate is at least one of an ozonated fat and/or oil, an ozonated animal-derived fat and/or oil, an ozonated beef fat and/or oil, an ozonated plant-derived fat and/or oil, an ozonated grape leaf oil, a non-ozonated fat and/or oil, a non-ozonated animal-derived fat and/or oil, a non-ozonated beef fat and/or oil, a non-ozonated plant-derived fat and/or oil, and a non-ozonated grape leaf oil.

18. A method of preventing transmission of HIV comprising:
injecting into at least one of an anatomical feature of a urethra, a seminal tract, a prostate gland, a vagina, and a rectum of an HIV-infected or non-infected human an amount of a first composition comprising an organomercury compound, ascorbic acid and/or an ascorbic acid salt, and citric acid and/or a citric acid salt so that bodily fluids within said anatomical feature contain said composition to reduce the amount of any HIV virus present in the bodily fluids to thereby reduce HIV transmission.

19. The method of claim 18, wherein:
the organomercury compound is thimerosal.

20. The method of claim 18, wherein:
the composition facilitates lowering the pH of the bodily fluids to a pH of from 4.5 or less.
